(19) United States
(12) Patent Application Publication
(10) Pub. No.: US 2005/0208118 A1
(43) Pub. Date: Sep. 22, 2005

(54) Preparations of encapsulated bioavailable chelating agents for detoxifying humans and animals

(76) Inventor: Arnold C. Takemoto, Scottsdale, AZ (US)

Correspondence Address: GREGORY SHEN, 4959 LORRAINE DRIVE, SAN DIEGO, CA, 92115, US

(21) Appl. No.: 10/804,264

(22) Filed: Mar. 18, 2004

(51) Int. Cl. A61K 9/127

(52) U.S. Cl. 424/450

(57) ABSTRACT

This invention provides, in non-limiting embodiments, novel preparations of chelating agents encapsulated in micelles or liposomes comprising the triple combination of: 1) micelles or liposomes comprising alpha lipoic acid or a derivative thereof and 2) micelles or liposomes comprising a chelating agent, such as EDTA; and furthermore, in different embodiments, optionally 3) magnesium chloride. The micelles or liposomes may be comprised of what have been termed “essential phospholipids”.

### FIELD OF THE INVENTION

This invention relates to preparations comprising chelating agents that are serviceable for the heavy metal detoxification of humans and animals and that can, in non-limiting fashion, be administrated orally, parenterally, or transdermally. In non-limiting exemplifications, this invention provides novel preparations of chelating agents encapsulated in micelles or liposomes comprising the triple combination of 1) micelles or liposomes comprising alpha lipoic acid and 2) micelles or liposomes comprising EDTA or other chelators; and furthermore, in different embodiments, 3) magnesium chloride is optionally an additional ingredient in these novel preparations.

### RELATED APPLICATIONS

Priority is claimed to provisional application (Ser. No. not yet assigned, filed Mar. 17, 2004, entitled: Detoxification and chelating preparations that can be administrated orally, parenterally, and transdermally, and related methods).

### BACKGROUND OF THE INVENTION

Toxicity and Poisoning.

Heavy metal poisoning is a serious medical problem that is receiving even more emphasis in recent years as the ability to detect toxic metals as well as the ability to understand the detrimental affects associated therewith have progressed compared to the past. Furthermore, it is known that toxic heavy metals such as lead and mercury may very easily enter the body as a consequence of, to name a few examples, accumulated exposure, accidents, environmental pollution, and oral consumption (e.g. food or paint). For example, exposures to lead and mercury are wide-spread and well documented. Poisoning from excessive concentrations of substances that would other wise be beneficial at lower concentrations is also known; e.g. iron poisoning has been reported. Arsenic can get into the body, e.g. as a result of industrial pollution. Also of concern are radioactive toxic heavy metals that pose an additional problem due to their radioactivity. These must be eliminated as quickly as possible, because the ionizing radiations of the radioactive metals pose the risk of tumor induction from their radioactive ionization, including by altering DNA. Toxic heavy metals are also known to concentrate in various organs of the body. Plutonium, for example, usually deposits in the liver, and it is known that as much as 30 to 60% or more of an administered amount of plutonium will oftentimes deposit in the liver. The toxic heavy metal, plutonium in this example, remains in the organ and is only very slowly removed, thereby increasing the potential for tumors.

Summary of Challenges with Traditional Treatment.

1) I.v. chelation is expensive, time-consuming, and has poor patient compliance.

2) Traditional oral chelation therapies are cheaper, but they are relatively ineffective at their intended purposes, and, at higher doses, are accompanied by side effects. For example, the oral administration of chelating agents by traditional approaches is problematic not only because their poor absorption and bioavailability prevents them from reaching the bodily stores of toxins and heavy metals, but furthermore they can chelate beneficial substances in the digestive tract.

3) Using traditional therapies, neither parenterally (e.g. by i.v.) nor orally administered chelating agents are able to enter the intracellular compartments where toxins and heavy metals are also present. Traditional therapies for the parenteral administration of chelating agents using physiologically compatible aqueous solutions (e.g. saline, Ringer's solution, etc.), fail to cause absorption of lipid soluble agents, because of inherent solubility problems.

1) Challenges with i.v. Chelation Therapies.

Heavy metal detoxification can be accomplished using i.v. chelation with ingredients such as EDTA; this approach has been documented to be effective and safe, and EDTA was approved by the FDA for this use in the 1950's. The ability of i.v. chelation therapy to diminish and even dissolve arterial plaques has also been reported. However, i.v. chelation is very expensive and time-consuming, typically requiring a patient make a series of 20 to 50 visits to a physician's office or hospital (at least 30 visits are typically required), with each visit often taking from 3-4 hours, during which time the patient is typically seated, and costing up to $100 or more per visit.

2) Challenges with Orally Administered Chelation Therapies.

Oral chelation products are commercially available, and they are marketed as much less expensive alternatives to i.v. chelation therapies. However, EDTA is very poorly absorbed when administered by mouth; and the general consensus is that typically only about five percent is absorbed. Although even that small amount does remove lead from the body, it also been reported to increases the absorption of lead.

Other serious potential problems have been reported as well. For example, it has been reported that the unabsorbed 95 percent of EDTA that remains within the digestive tract, mixes with undigested food and nutrients while passing on out of the body in stool. This unabsorbed EDTA tightly binds to and blocks absorption of many essential nutritional trace elements as it passes through, thereby potentially blocking the uptake of important nutrients such as zinc, manganese, chromium, vanadium, copper, chromium, molybdenum and other essential nutrients, causing deficiencies.

When a chelator such as EDTA enters the body, either by mouth or intravenously, it could possibly remove 10 to 20 times more of the essential nutritional trace elements (such as zinc and manganese) than it does the undesired heavy metals or toxins that are deleterious. When given intravenously, thus bypassing any absorption problems, a full therapeutic treatment of EDTA can be completed with 20 to 50 daily doses. The replenishment of the lost essential trace elements by dietary supplementation can then take place during the remaining 315+days of the year after the treatment, when the exogenously administered chelating agent(s) such as EDTA have been excreted or eliminated, and are not present to interfere. Because such a small amount is absorbed by mouth, oral EDTA is often given every day, but for up to 20 times or more as long, to accumulate what is alleged to be an effective dose, and there is no interim opportunity to replenish the essential nutrients that are being continuously blocked and depleted during the chelation therapy.

Thus, the daily administration of chelating agents such as EDTA by mouth may cause progressive deficiencies of zinc, manganese and other essential trace nutrients, which are an essential part of the body's antioxidant defenses. For example, the activity of superoxide dismutase (SOD), a very important intracellular antioxidant, depends on zinc and manganese. By inactivating antioxidant enzymes, the daily intake of chelation agents by mouth may actually worsen the condition of the patients being treated.

Intravenous chelation therapy has been reported to stimulate the release of parathyroid hormone (parathormone) in a pulsatile manner, but orally administered chelation therapies, such as with EDTA, have not. Thus, if that mechanism of action is important to achieve the intended benefit, oral EDTA cannot achieve the goal.

Attempts have been reported to increase the amount of chelating agents that are used in an oral chelation therapy to match the levels that can be achieved when they are administered intravenously. However, there are many side effects that prevent this approach from being used.

3) Challenges with Both Oral and i.v. Chelation Therapies.

The use of chelating agents for the removal of toxic heavy metals is based on their ability to form stable, nonionic, soluble and readily excretable complexes with the metal molecules in the tissues. They have proven valuable because they, in themselves, have a very low toxicity, are able to form soluble, excretable metal chelates within a body, and resist degradation by cell metabolites. However, the serious limitation for the use of chelating agents is that, when introduced into a body, they exist as hydrated anions in the blood plasma These anions are unable to penetrate cellular membranes. Therefore, only extracellularly deposited toxic metals can be complexed by the chelating agents and removed from the body, whereas intracellularly deposited metals are not complexed by the chelating agent and therefore are not readily removed. Attempts have been made in the past to increase the penetration of chelating agents through cellular membranes such as by the esterification of polyaminopolycarboxylic acids, but these efforts have met with limited success because of the insolubility and toxicity of the esterified compounds.

Thus, chelators such as EDTA typically remain extracellularly or outside of cells. By way of illustration, orally administered EDTA reaches only very low concentrations outside cell surfaces in the body and for brief periods of time, while intravenous infusions result in much higher levels, and can be maintained for several hours. However, intravenously administered EDTA can only chelate unwanted metals and toxins, if, e.g. they travel out of cell walls by diffusion. In contrast, this is not believed to occur to a significant extent—if at all—with chelators such as EDTA when taken by mouth. In sum, neither traditional approach achieves significant intracellular levels of chelating agents, and is thus unable to readily exert its actions intracellularly.

The preparations of the present invention comprise antioxidants that have effects that may be additive or synergistic to the effects of chelators such as EDTA; however, these antioxidants may be lipophilic. Because many parenterally suitable fluids such as saline, dextran, blood, stabilized hemoglobin solutions, etc., are all aqueous solutions, a problem with therapies based on lipid soluble antioxidants, such as .alpha.-lipoic acid, is the poor water solubility of these ingredients. The solubility may be enhanced by adding benzyl alcohol or DMSO, but such solvents introduce additional side effects.

Previous methods of delivering lipophilic antioxidants that involved solubilizing the antioxidant in solvents such as benzyl alcohol, DMSO, or other chemicals not only have the potential to introduce new toxicities, e.g. they may exacerbate microvascular injury, but the presence of these solvents confuses the interpretation of any protocol designed to evaluate antioxidant effects.

This invention provides novel solutions to these and other problems.

### SUMMARY OF THE INVENTION

It is an object of the present invention to provide a method for transferring at least two ingredients, comprising an antioxidant and a chelating agent, across a cellular membrane.

Another object of the present invention is to provide a means for introducing at least two ingredients, comprising an antioxidant and a chelating agent, into the interior of a cell.

It is another object of the present invention to provide a method for introducing at least two ingredients, comprising an antioxidant and a chelating agent, into the interior of a cell of a living organism by introducing the at least two ingredients to the organism and carrying it to the cell in the blood stream. In a preferred but non-limiting aspect, the at least two ingredients are introduced by oral administration.

Another object of the present invention is to provide a method for the removal of intracellularly deposited toxic heavy metals.

Still another object of the present invention is to provide a therapy method for toxic heavy metal poisoning whereby both intracellularly deposited toxic heavy metals as well as extracellularly deposited toxic heavy metals can be removed from the body. In separate aspect, said body is a human body or an animal body (e.g. a pet or other raised animal).

### TERMS

Biologically active and bioactive are used interchangeably, and can refer to in vitro as well as to in vivo situations.

Physiological solutions suitable for intravenous injection include: e.g. Saline. In lieu of normal saline, other pharmaceutically acceptable solutions may be utilized including, but not limited to, 0.9% saline solution, 5% dextrose solution, lactated Ringer's solution, 5% dextrose in lactated Ringer's solution, dextrose-saline combinations, albumin-containing solutions, dextran, dextran-saline combinations, etc.

POEBACA: preparation(s) of encapsulated bioavailable chelating agents(s). Both plural and singular meanings are included.

POEBACAI: ingredient(s) for making up (a) preparation(s) of encapsulated bioavailable chelating agents(s). POEBACAI can exist in encapsulated form or in nonencapsulated form (e.g. a pre-encapsulated stage). Both plural and singular meanings are included.

1 ounce (oz.)=28.3495231 grams (gm)

128 ounces=1 gallon

### DETAILED DESCRIPTION OF THE INVENTION

Ingredients.

This invention provides novel preparations of encapsulated bioavailable chelating agents (POEBACA), wherein in each of different preferred embodiments a POEBACA is comprised of the following ingredients (or PEOBACAI), for which non-limiting examples are listed in Table 1:

a) one or more members selected from Group A (e.g. alpha lipoic acid);

b) one or more members selected from Group B (e.g. EDTA);

c) one or more members selected from Group C (e.g. lecithin);

d) optionally, in separate embodiments, one or more members selected from Group D (e.g. magnesium chloride);

e) optionally, in separate embodiments, one or more members selected from Group E (glutathione);

f) optionally, in separate embodiments, one or more members selected from Group F (e.g. vinpocetine);

g) optionally, in separate embodiments, one or more members selected from Group G (e.g. nitrogen gas);

wherein the ingredients are prepared in a manner that provides the encapsulation of a significant fraction of one or more ingredient(s) into liposomes or micropsheres.

Preferred Numbers of Group A Members (e.g. Alpha Lipoic Acid).

According to this invention, separate preferred embodiments of “preparations of encapsulated bioavailable chelating agents” (i.e. POEBACA) are provided herein, each of which is comprised of at least a minimum number of members, i.e. “n” member(s), selected from Group A, where n=1, 2, 3, . . . , 100, including every integer value within the range of 1 to 100. Thus, there are at least 100 embodiments of POEBACA, differing in that the minimum number of members selected from Group A that is contained in each embodiment ranges from one to one hundred (including every integer value in between), i.e. at least one, at least two, at least three, at least four, . . . , and up to at least 100. Thus, one preferred embodiment of this invention provides a POEBACA comprised of at least one member selected from Group A; another preferred embodiment of this invention provides a POEBACA comprised of at least two members selected from Group A; another preferred embodiment of this invention provides a POEBACA comprised of at least three members selected from Group A; etc. ; another preferred embodiment of this invention provides a POEBACA comprised of at least one hundred members selected from Group A; for convenience these are referred to as preferred embodiments A1 to A100, and these separate embodiments are intended to be the subject matter of separate claims according to this invention.

Preferred Numbers of Group B Members (e.g. EDTA).

According to this invention, separate preferred embodiments of “preparations of encapsulated bioavailable chelating agents” (i.e. POEBACA) are provided herein, each of which is comprised of at least a minimum number of members, i.e. “n” member(s), selected from Group B, where n=1, 2, 3, . . . , 100, including every integer value within the range of 1 to 100. Thus, there are at least 100 embodiments of POEBACA, differing in that the minimum number of members selected from Group B that is contained in each embodiment ranges from one to one hundred (including every integer value in between), i.e. at least one, at least two, at least three, at least four, . . . , and up to at least 100. Thus, one preferred embodiment of this invention provides a POEBACA comprised of at least one member selected from Group B; another preferred embodiment of this invention provides a POEBACA comprised of at least two members selected from Group B; another preferred embodiment of this invention provides a POEBACA comprised of at least three members selected from Group.B; etc. ; another preferred embodiment of this invention provides a POEBACA comprised of at least one hundred members selected from Group B; for convenience these are referred to as preferred embodiments B1 to B100, and these separate embodiments are intended to be the subject matter of separate claims according to this invention.

Preferred Numbers of Group C Members (e.g. Lecithin).

According to this invention, separate preferred embodiments of “preparations of encapsulated bioavailable chelating agents” (i.e. POEBACA) are provided herein, each of which is comprised of at least a minimum number of members, i.e. “n” member(s), selected from Group C, where n=1, 2, 3, . . . , 100, including every integer value within the range of 1 to 100. Thus, there are at least 100 embodiments of POEBACA, differing in that the minimum number of members selected from Group C that is contained in each embodiment ranges from one to one hundred (with every integer value in between), i.e. at least one, at least two, at least three, at least four, . . . , and up to at least 100. Thus, one preferred embodiment of this invention provides a POEBACA comprised of at least one member selected from Group C; another preferred embodiment of this invention provides a POEBACA comprised of at least two members selected from Group C; another preferred embodiment of this invention provides a POEBACA comprised of at least three members selected from Group C; etc. ; another preferred embodiment of this invention provides a POEBACA comprised of at least one hundred members selected from Group C; for convenience these are referred to as preferred embodiments C1 to 100, and these separate embodiments are intended to be the subject matter of separate claims according to this invention.

Preferred Numbers of Group D Members (e.g. Magnesium Chloride).

According to this invention, separate preferred embodiments of “preparations of encapsulated bioavailable chelating agents” (i.e. POEBACA) are provided herein, each of which is comprised of at least a minimum number of members, i.e. “n” member(s), selected from Group D, where n=1, 2, 3, . . . , 20, including every integer value within the range of 1 to 20. Thus, there are at least 20 embodiments of POEBACA, differing in that the minimum number of members selected from Group D that is contained in each embodiment ranges from one to twenty (including every integer value in between), i.e. at least one, at least two, at least three, at least four, . . . , and up to at least 20. Thus, one preferred embodiment of this invention provides a POEBACA comprised of at least one member selected from Group D; another preferred embodiment of this invention provides a POEBACA comprised of at least two members selected from Group D; another preferred embodiment of this invention provides a POEBACA comprised of at least three members selected from Group D; etc. ; another preferred embodiment of this invention provides a POEBACA comprised of at least twenty members selected from Group D; for convenience these are referred to as preferred embodiments D1 to D20, and these separate embodiments are intended to be the subject matter of separate claims according to this invention.

Preferred Numbers of Group E Members (e.g. Glutathione).

According to this invention, separate preferred embodiments of “preparations of encapsulated bioavailable chelating agents” (i.e. POEBACA) are provided herein, each of which is comprised of at least a minimum number of members, i.e. “n” member(s), selected from Group E, where n=1, 2, 3,. . . , 20, including every integer value within the range of 1 to 20. Thus, there are at least 20 embodiments of POEBACA, differing in that the minimum number of members selected from Group E that is contained in each embodiment ranges from one to twenty (including every integer value in between), i.e. at least one, at least two, at least three, at least four, . . . , and up to at least 20. Thus, one preferred embodiment of this invention provides a POEBACA comprised of at least one member selected from Group E; another preferred embodiment of this invention provides a POEBACA comprised of at least two members selected from Group E; another preferred embodiment of this invention provides a POEBACA comprised of at least three members selected from Group E; etc. ; another preferred embodiment of this invention provides a POEBACA comprised of at least twenty members selected from Group E; for convenience these are referred to as preferred embodiments E1 to E20, and are intended to be claimed subject matter according to this invention.

Preferred Numbers of Group F Members (e.g. Vinpocetine).

According to this invention, separate preferred embodiments of “preparations of encapsulated bioavailable chelating agents” (i.e. POEBACA) are provided herein, each of which is comprised of at least a minimum number of members, i.e. “n” member(s), selected from Group F, where n=1, 2, 3, . . . , 20, including every integer value within the range of 1 to 20. Thus, there are at least 20 embodiments of POEBACA, differing in that the minimum number of members selected from Group F that is contained in each embodiment ranges from one to twenty (including every integer value in between), i.e. at least one, at least two, at least three, at least four, . . . , and up to at least 20. Thus, one preferred embodiment of this invention provides a POEBACA comprised of at least one member selected from Group F; another preferred embodiment of this invention provides a POEBACA comprised of at least two members selected from Group F; another preferred embodiment of this invention provides a POEBACA comprised of at least three members selected from Group F; etc. ; another preferred embodiment of this invention provides a POEBACA comprised of at least twenty members selected from Group F; for convenience these are referred to as preferred embodiments F1 to F20, and these separate embodiments are intended to be the subject matter of separate claims according to this invention.

Preferred Numbers of Group G Members (e.g. Nitrogen Gas).

According to this invention, separate preferred embodiments of “preparations of encapsulated bioavailable chelating agents” (i.e. POEBACA) are provided herein, each of which is comprised of at least a minimum number of members, i.e. “n” member(s), selected from Group G, where n=1, 2, 3, . . . , 20, including every integer value within the range of 1 to 20. Thus, there are at least 20 embodiments of POEBACA, differing in that the minimum number of members selected from Group G that is contained in each embodiment ranges from one to twenty (including every integer value in between), i.e. at least one, at least two, at least three, at least four, . . . , and up to at least 20. Thus, one preferred embodiment of this invention provides a POEBACA comprised of at least one member selected from Group G; another preferred embodiment of this invention provides a POEBACA comprised of at least two members selected from Group G; another preferred embodiment of this invention provides a POEBACA comprised of at least three members selected from Group G; etc. ; another preferred embodiment of this invention provides a POEBACA comprised of at least twenty members selected from Group G; for convenience these are referred to as preferred embodiments G1 to G20, and these separate embodiments are intended to be the subject matter of separate claims according to this invention.

Preferred Numbers of Members from Groups A Through G.

This invention further provides the additional preferred aspects that result from all the possible combinations and permutations of the preferred embodiments of A1 to A100, B1 to B100, C1 to C100, D1 to D20, E1 to E20, F1 to F20, and G1 to G20. By way of illustration, (100 preferred embodiments corresponding to A1 to A100)×(100 preferred embodiments corresponding to B1 to B 100)×(100 preferred embodiments corresponding to C1 to C100)×(20 preferred embodiments corresponding to D1 to D20)×(20 preferred embodiments corresponding to E1 to E20)×(20 preferred embodiments corresponding to F1 to F100)×(100 preferred embodiments corresponding to G1 to G20)=160,000,000,000 or one hundred and sixty billion preferred aspects, and these separate aspects are intended to be the subject matter of separate claims according to this invention.

Preferred Amounts of Ingredients.

Furthermore, the relative amounts of each ingredient that can comprise a POEBACA according to this invention are illustrated in Table 2. In separate embodiments, this invention provides all the physically possible combinations and permutations of ingredient amounts that listed in Table 2. Thus, this invention provides that the relative amounts of these ingredients can vary (as illustrated in Table 2), yielding additional aspects. Therefore, when considering the claim limitations regarding the relative amount of ingredients, the number of preferred embodiments is greater, by orders of magnitude, than 160,000,000,000 or one hundred and sixty billion preferred embodiments that don't specify amounts of ingredients, and all these preferred embodiments are intended to be the subject matter of separate claims according to this invention.

TABLE 1
Ingredients

| Group | Group Members (Non-limiting examples are listed for each group) |
|---|---|
| A | Antioxidants and hydrophobic ingredients |
| | R-(+)-.alpha.-lipoic acid (substantially enantiomerically pure), S-(−)-.alpha.- |
| | lipoic acid (substantially enantiomerically pure), R/S-.alpha.-lipoic acid |
| | (racemic mixture), R/S-.gamma.-lipoic acid (racemic micture), other isomers of |
| | alpha lipoic acid, derivatives of alpha lipoic acid (such as the dihydro version of |
| | these alpha lipoic acid isomers, also known as dihydrolipoic acid or DHLA), |
| | animal and vegetable oils, hydrocarbon oils, ester oils, silicone oils, higher fatty |
| | acids, higher alcohols, sunscreening agents, vitamins, ferulic acid |
| B | Chelators |
| | EDTA, EGTA, DPTA, TTHA, HEDHA, NOTA, DOTA, HEDTA, other |
| | polyaminopolycarboxylic acids, iminodiacetic acid (IDA), cyclam, |
| | penicillamine, dimercaptosuccinic acid, tartrate, thiomalic acid, crown ethers, |
| | nitrilotriacetatic acid (NTA), 3,6-dioxaoctanedithioamide, 3,6- |
| | dioxaoctanediamide, salicyladoximine, dithio-oxamide, 8-hydroxyquinoline, |
| | cupferron, 2,2′-thiobis(ethyl acetoacetate), 2,2′-dipyridyl, and derivatives |
| | thereof |
| C | Phospholipids, lipids and fatty acids |
| | lecithin, phophatidylcholine, phosphatidylserine, phosphatidylethanolamine, |
| | dilinoleylphosphatidylcholine, lysolipids, dipalmitoylphosphatidylcholine, |
| | distearoylphosphatidylcholine, phosphatidylcholine, phosphatidic acid, |
| | sphingomyelin, cholesterol, cholesterol sulfate, cholesterol hemisuccinate, |
| | tocopherol hemisuccinate, phosphatidylethanolamine, phosphatidylinositol, |
| | fatty acids (e.g. palmitic acid, stearic acid, oleic acid, linolenic acid, limoleic |
| | acid, etc.), glycosphingolipids, glucolipids, glycolipids, sulphatides, lipids |
| | bearing sulfonated mono-, di-, oligo- or polysaccharides, lipids with ether and |
| | ester-linked fatty acids, triglycerides, lipoproteins (high or low density), |
| | cholesterol, and other lipids and polymerized lipids. |
| D | Magnesium Salts |
| | Magnesium chloride, Magnesium Gluconate, Magnesium Carbonate, Calcium |
| | Magnesium Citrate, Magnesium Sulfate |
| E | Sulfur-Containing Amino Acids, Sulfur-Containing Peptides, Sulfur-Containing |
| | Proteins |
| | Glutathione, methionine, cysteine |
| F | Plant alkaloids (e.g. vinpocetine, vincamine), coenzyme Q10, and analogues |
| | coenzyme Q10 (e.g. idebenone) |
| G | Gaseous ingredient |
| | Nitrogen gas, oxygen gas, atmospheric air, gaseous mixtures containing |
| | nitrogen gas, gaseous mixtures containing oxygen gas. |

TABLE 2
Amounts of ingredients (normalized to 2 oz or approximately 56 grams)

| Group | Example 1 | Example 1 | Preferred amounts intended for protection |
|---|---|---|---|
| (with example of a group | (Absolute | (Relative | according to this invention (both |
| member) | amount, mg) | amount, %) | individually and collectively as a group) |
| A (e.g. alpha lipoic acid) | 100.0 mg | 0.17 | From about 0.01 mg to about 20,000 mg |
| | | | inclusive, including specifically each |
| | | | increment of about 0.01 mg within this |
| | | | range. |
| B (e.g. EDTA) | 1,000.0 mg | 1.7 | From about 0.01 mg to about 30,000 mg |
| | | | inclusive, including specifically each |
| | | | increment of about 0.01 mg within this |
| | | | range. |
| C (e.g. lecithin) | 30,000.0 mg | 50.0 | From about 0.01 mg to about 40,000 mg |
| | | | inclusive, including specifically each |
| | | | increment of about 0.01 mg within this |
| | | | range. |
| D (e.g. magnesium chloride) | 150.0 mg | 0.26 | From about 0.01 mg to about 10,000 mg |
| | | | inclusive, including specifically each |
| | | | increment of about 0.01 mg within this |
| | | | range. |
| E (e.g. glutathione) | 1,000 mg | 1.7 | From about 0.01 mg to about 10,000 mg |
| | | | inclusive, including specifically each |
| | | | increment of about 0.01 mg within this |
| | | | range. |
| F (e.g. vinpocetine) | 100 mg | 0.17 | From about 0.01 mg to about 10,000 mg |
| | | | inclusive, including specifically each |
| | | | increment of about 0.01 mg within this |
| | | | range. |

Example 1.
Other ingredients: Water (30-40%), Ethanol (5-15%), Gum Arabic (0.5-2%), Flavorings (0-5%).

In Table 2 the relative amounts of each ingredient (POEBACAI) have been expressed in the context of a 2 ounce dose. This is for convenience and consistency, but in separate embodiments this invention provides that that dosages or other sizes can be prepared and administered, particularly ranging, by way of non-limiting exemplification, from about 0.1 ounce to about 128 ounces (or one gallon), including every 0.1 ounce increment in between.

Preferred Amount(s) of Group A Members (e.g. Alpha Lipoic Acid).

This invention provides separate embodiments wherein per 2 ounces the total amount of ingredient(s) from Group A (e.g. alpha lipoic acid) collectively is preferably from about 0.01 mg to about 20,000 mg inclusive, including specifically each increment of about 0.01 mg within this range. Furthermore, this invention provides separate embodiments wherein per 2 ounces the total amount of each specific Group A ingredient(s) individually is preferably from about 0.01 mg to about 20,000 mg inclusive, including specifically each increment of about 0.01 mg within this range.

Thus; by way of illustration:

1) in one embodiment, this invention provides preparations of encapsulated bioavailable chelating agents (i.e. POEBACA) wherein the total amount of Group A members (e.g. alpha lipoic acid) is preferably 0.01 mg;

2) in another embodiment, this invention provides preparations of encapsulated bioavailable chelating agents (i.e. POEBACA) wherein the total amount of Group A members (e.g. alpha lipoic acid) is preferably 0.02 mg;

3) in another embodiment, this invention provides preparations of encapsulated bioavailable chelating agents (i.e. POEBACA) wherein the total amount of Group A members (e.g. alpha lipoic acid) is preferably 0.03 mg; etc. ; and

2,000,000) in another embodiment, this invention provides preparations of encapsulated bioavailable chelating agents (i.e. POEBACA) wherein the total amount of Group A members (e.g. alpha lipoic acid) is preferably 20,000 mg.

Thus, there are at least 2,000,000 preferred embodiments. This is illustrated in Table 2.

Preferred Amount(s) of Group B Members (e.g. EDTA).

This invention provides separate embodiments wherein per 2 ounces the total amount of ingredient(s) from Group B (e.g. EDTA) collectively is preferably from about 0.01 mg to about 30,000 mg inclusive, including specifically each increment of about 0.01 mg within this range. Furthermore, this invention provides separate embodiments wherein per 2 ounces the total amount of each specific Group B ingredient(s) individually is preferably from about 0.01 mg to about 30,000 mg inclusive, including specifically each increment of about 0.01 mg within this range.

Preferred Amount(s) of Group C Members (e.g. Lecithin).

This invention provides separate embodiments wherein per 2 ounces the total amount of ingredient(s) from Group C (e.g. lecithin) collectively is preferably from about 0.01 mg to about 40,000 mg inclusive, including specifically each increment of about 0.01 mg within this range. Furthermore, this invention provides separate embodiments wherein per 2 fluid ounces the total amount of each specific Group C ingredient(s) individually is preferably from about 0.01 mg to about 40,000 mg inclusive, including specifically each increment of about 0.01 mg within this range.

Preferred Amount(s) of Group D Members (e.g. Magnesium Chloride).

This invention provides separate embodiments wherein per 2 ounces the total amount of ingredient(s) from Group D (e.g. magnesium chloride) collectively is preferably from about 0.01 mg to about 10,000 mg inclusive, including specifically each increment of about 0.01 mg within this range. Furthermore, this invention provides separate embodiments wherein per 2 ounces the total amount of each specific Group D ingredient(s) individually is preferably from about 0.01 mg to about 10,000 mg inclusive, including specifically each increment of about 0.01 mg within this range.

Preferred Amount(s) of Group E Members (e.g. Glutathione).

This invention provides separate embodiments wherein per 2 ounces the total amount of ingredient(s) from Group E (e.g. glutathione) collectively is preferably from about 0.01 mg to about 10,000 mg inclusive, including specifically each increment of about 0.01 mg within this range. Furthermore, this invention provides separate embodiments wherein per 2 fluid ounces the total amount of each specific Group E ingredient(s) individually is preferably from about 0.01 mg to about 10,000 mg inclusive, including specifically each increment of about 0.01 mg within this range.

Preferred Amount(s) of Group F Members (e.g. Vinpocetine).

This invention provides separate embodiments wherein per 2 ounces the total amount of ingredient(s) from Group F (e.g. vinpocetine) collectively is preferably from about 0.01 mg to about 10,000 mg inclusive, including specifically each increment of about 0.01 mg within this range. Furthermore, this invention provides separate embodiments wherein per 2 ounces the total amount of each specific Group F ingredient(s) individually is preferably from about 0.01 mg to about 10,000 mg inclusive, including specifically each increment of about 0.01 mg within this range.

Preferred Percentages of Encapsulated Group G Members (e.g. Nitrogen Gas).

This invention provides separate embodiments wherein one or more gases may be contained in a percentage of the liposomes or micropsheres in a POEBACA. In separate embodiments, the percent of liposomes or micropsheres that contains a gas is from about 1% to about 100%, including every integer value in between.

Preferred Methods of Administration.

This invention provides POEBACA that can be administered by several routes, including intravenous, topical, and oral. Furthermore, in separate embodiments, this invention provides forms of POEBACA that can be administered by inoculation or injection, (e.g., intraperitoneal, intramuscular, subcutaneous, intra-aural, intra-articular, intra-mammary, etc.), topical application (e.g., on areas, such as eyes, ears, skin or on afflictions such as wounds, burns, etc.), and by absorption through epithelial or mucocutaneous linings (e.g. vaginal and other epithelial linings, gastrointestinal mucosa, etc.). Methods are known for making POEBACA containing liposomes that are suitable for each of these methods of administration as well as other methods of administration that are know in the art. For example, in preferred embodiments, this invention provides POEBACA in liquid forms that can be administered orally. The POEBACA can be also prepared as capsules, tablets, pellets (e.g. for animal consumption), suppositories, or creams and ointments. The POEBACA can be also prepared as physiological solutions suitable for i.v. administration or other parenteral administration.

In as many separate aspects, this invention also provides all the possible combinations of ingredient quantities that are possible (e.g. the total of all the ingredients or POEBACAI does not surpass 100% of the relevant total dosage of the POEBACA, and admixing or solubility limitations are not exceeded).

Preferred Percentages of Ingredients that are Contained in Liposomes or Micropsheres.

In separate aspects, this invention also provides that a POEBACA may include ingredients (or POEBACAI) that are not contained in micropsheres or liposomes in addition to ingredients that are contained in liposomes, and that these ingredients may be the same or different substances.

In separate aspects, this invention also provides that for each ingredient (or POEBACAI) the percent that is contained in micropsheres or liposomes (in contrast to the percentage that is not contained in micropsheres or liposomes, but rather is in solution) may be from about 0.1% to about 100.0%, including every 0. 1% increment within this range. This provides at least about 1000 separate aspects that are intended for protection according to this invention.

In separate aspects, this invention also provides that in a single POEBACA, the micropsheres or liposomes may be fairly homogeneous in size or in content; alternatively they may be fairly heterogeneous in size or in content.

Preferred Group A Members (e.g. Alpha Lipoic Acid).

Group A members include: antioxidants, particularly hydrophobic antioxidants and other hydrophobic ingredients.

Group A members include, but are not limited to:

R-(+)-.alpha.-lipoic acid (substantially enantiomerically pure), S-(−)-.alpha.-lipoic acid (substantially enantiomerically pure), R/S-.alpha.-lipoic acid (racemic mixture), R/S-.gamma.-lipoic acid (racemic micture), other isomers of alpha lipoic acid, derivatives of alpha lipoic acid (such as the dihydro version of these alpha lipoic acid isomers, also known as dihydrolipoic acid or DHLA), animal and vegetable oils, hydrocarbon oils, ester oils, silicone oils, higher fatty acids, higher alcohols, sunscreening agents, vitamins, ferulic acid. Group A members also include, but are not limited to:

fatty acids, lysolipids, dipalmitoylphosphatidylcholine, distearoylphosphatidylcholine, phosphatidylcholine, phosphatidic acid, sphingomyelin, cholesterol, cholesterol sulfate, cholesterol hemisuccinate, tocopherol hemisuccinate, phosphatidylethanolamine, phosphatidylinositol, glycosphingolipids, glucolipids, glycolipids, sulphatides, lipids bearing sulfonated mono-, di-, oligo- or polysaccharides, lipids with ether and ester-linked fatty acids, and polymerized lipids.

Preferred Group B Members (e.g. EDTA).

Group B members include: chelators or chelating agents.

Group B members include, but are not limited to:

EDTA, EGTA, DPTA, TTHA, HEDHA, NOTA, DOTA, HEDTA, other polyaminopolycarboxylic acids, iminodiacetic acid (IDA), cyclam, penicillamine, dimercaptosuccinic acid, tartrate, thiomalic acid, crown ethers, nitrilotriacetatic acid (NTA), 3,6-dioxaoctanedithioamide, 3,6-dioxaoctanediamide, salicyladoximine, dithio-oxamide, 8-hydroxyquinoline, cupferron, 2,2′-thiobis(ethyl acetoacetate), 2,2′-dipyridyl, and derivatives thereof. According to this invention, other chelators that are members of Group B are provided herein or are otherwise known in the art and can serve as ingredients for this invention.

Preferred Group C Members (eg. Lecithin).

Group C members include: phospholipids, lipids and fatty acids.

Group C members include, but are not limited to:

lecithin, phophatidylcholine, phosphatidylserine, phosphatidylethanolamine, dilinoleylphosphatidylcholine, lysolipids, dipalmitoylphosphatidylcholine, distearoylphosphatidylcholine, phosphatidylcholine, phosphatidic acid, sphingomyelin, cholesterol, cholesterol sulfate, cholesterol hemisuccinate, tocopherol hemisuccinate, phosphatidylethanolamine, phosphatidylinositol, fatty acids (e.g. palmitic acid, stearic acid, oleic acid, linolenic acid, limoleic acid, etc.), glycosphingolipids, glucolipids, glycolipids, sulphatides, lipids bearing sulfonated mono-, di-, oligo- or polysaccharides, lipids with ether and ester-linked fatty acids, triglycerides, lipoproteins (high or low density), cholesterol, and other lipids and polymerized lipids.

Preferred Group D Members (e.g. Magnesium Chloride).

Group D members include: magnesium salts.

Group D members include, but are not limited to:

magnesium chloride, magnesium gluconate, magnesium carbonate, calcium magnesium citrate, magnesium sulfate, other salts of magnesium, and other forms of magnesium.

Preferred Group E Members (e.g. Glutathione).

Group E embers include: sulfur-containing amino acids, sulfur-containing peptides, sulfur-containing proteins, and other sulfur-containing substances.

Group E members include, but are not limited to:

magnesium chloride, magnesium gluconate, magnesium carbonate, calcium magnesium citrate, magnesium sulfate, other salts of magnesium, and other forms of magnesium

Preferred Group F Members (e.g. Vinpoctine).

Group F members include: Vinpocetine, vincamine, idebenone

Preferred Group G Members (e.g. Nitrogen Gas).

Group G members include: Nitrogen gas, atmospheric air, and other mixtures of gases that contain nitrogen, oxygen, mixtures of gases that contain oxygen, argon, and mixtures of gases that contain argon, etc.

Lipophilic Anti-oxidants (e.g. alpha lipoic acid). Alpha-lipoic acid, in addition to its non-toxicity and lipophilicity, has the advantage of being rapidly converted in tissues into its reduced form, dihydrolipoic acid (DHLA). DHLA also has potent antioxidant effects. Further, both .alpha.-lipoic acid and DHLA have been shown to disarm oxidants through a variety of mechanisms including free radical quenching, metal chelation, and regeneration of other common natural antioxidants.

In one embodiment, the present invention provides a lipophilic antioxidant in an aqueous physiological fluid, such as a resuscitation fluid by lipid encapsulation, e.g. by providing liposomal formation methods to form stable micellular solutions of .alpha.-lipoic acid or other lipophilic antioxidant(s).

The present invention seeks to overcome previous limitations by solubilizing .alpha.-lipoic acid in aqueous solution without the use of solvents such as harsh organic solvents. .alpha.-lipoic acid and other antioxidants are rendered soluble in aqueous solutions by the use of liposomal formation processes, such as ultrasonication. Because the .alpha.-lipoic molecule contains a polar (water soluble) carboxy-acid group and a non-polar, lipid soluble chain of carbon and sulfur atoms, the molecule is amphipathic, i.e., it has the ability to form micelles. Micelles may be formed in aqueous solution if a molecule possesses both polar and non-polar groups. After ultrasonication the polar, a number of the water soluble ends of the .alpha.-lipoic acid molecule are on the outside of aggregations of .alpha.-lipoic acid. A number of the non-polar, lipid soluble tails are directed inward forming a tiny droplet, a micelle, which is water soluble. Ultrasonication of amphipathic molecules into micelles such as can be done with .alpha.-lipoic acid also has the possibility of creating mixed micelles. In this manner a mixture of .alpha.-lipoic acid with other antioxidants, which may not have the ability to form micelles alone for lack of any polar group, can be contained within a micelle of .alpha-lipoic acid. In this way, mixed micelles containing .alpha.-lipoic acid and purely non-polar but highly lipid soluble antioxidants can be used to convey antioxidants to the tissues.

There are numerous other clinical conditions besides hemorrhagic shock which have as their final common pathway oxidant-inducing injury to tissues which can be treated and/or prevented with the inventive solutions.

Chelating Agents

According to this invention, the polyaminopolycarboxylic acid, EDTA (ethylene-diaminetetraacetic acid) is provided as a chelating agent for removing toxins such as heavy metals. Additionally, a related polyaminopolycarboxylic acid, diethylenetriaminepentaacetic acid (DTPA) is also provided as a chelating agent that has been shown to have an ability to remove various heavy metals.

According to this invention, EGTA (ethyleneglycol-bis[.beta.-aminoethyl ether]-N,N′-tetra-acetic acid) is also provided as chelating agent. EGTA is more specific for particular substances such as calcium when compared to other substances such as magnesium, and thus may be used as a preferred ingredient when it is desirable to chelate calcium (e.g. as is found in arterial plaques, and thus for diminishing arterial plaques) more than for chelating magnesium.

DMSA (dimercaptosuccinic acid) is one effective oral chelating agent that is absorbed orally, and is more effective at chelating particular substances such as mercury, lead, and arsenic in comparison to other substances; and thus DMSA may be used as a preferred ingredient when it is desirable to chelate mercury lead and arsenic (such for the detoxification of poisoning from lead or mercury r arsenic) more than for chelating other substances.

According to this invention, other useful chelating agents are also provided, including diethylenetriamine-pentaacetic acid (DTPA), triethylenetetraaminehexaacetic acid (TTHA), N-hydroxyethylenediaminehexaacetic-acid (HEDHA), 1,4,7-triazacyclononane-N,N′,N″-triacetic acid (NOTA), 1,4,7,10-tetraa cyclododecane-N,N′,N″,N′″-tetraacetic acid (DOTA), and N′hydroxyethylenediamine-N,N,N′-triacetic acid (HEDTA).

According to this invention, preferred chelating agents also include iminodiacetic acid (IDA), cyclam, penicillamine, dimercaptosuccinic acid, tartrate, thiomalic acid, crown ethers, nitrilotriacetatic acid (NTA), 3,6-dioxaoctanedithioamide, 3,6-dioxaoctanediamide, salicyladoximine, dithio-oxamide, 8-hydroxyquinoline, cupferron, 2,2′-thiobis(ethyl acetoacetate), 2,2′-dipyridyl. IDA is a preferred chelating headgroup which is selective for copper ions.

Preferable chelators for use in the present invention include, but are not limited to, ethylenediamine-N,N,N′,N′-tetraacetic acid (EDTA); the disodium, trisodium, tetrasodium, dipotassium, tripotassium, dilithium and diammonium salts of EDTA; the barium, calcium, cobalt, copper, dysprosium, europium, iron, indium, lanthanum, magnesium, manganese, nickel, samarium, strontium, and zinc chelates of EDTA; trans-1,2-diaminocyclohexane-N,N,N′,N′-tetraaceticacid monohydrate; N,N-bis(2-hydroxyethyl)glycine; 1,3-diamino-2-hydroxypropane-N,N,N′,N′-tetraa cetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); O,O′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephosphoric acid); 7,19,30-trioxa-1,4,10,13,16,22,27,33-octaazabicyclo[11,11,11] pentatriacontane hexahydrobromide; and triethylenetetraine-N,N,N′,N″,N′″,N′″-hexaacetic acid. It is contemplated that any chelator which binds barium, calcium, cerium, cobalt, copper, iron, magnesium, manganese, nickel, strontium, or zinc will be acceptable for use in the present invention.

More preferably, the chelators for use in conjunction with the present invention may include ethylenediamine-N,N,N′,N′-tetraacetic acid (EDTA); the disodium, trisodium, tetrasodium, dipotassium, tripotassium, dilithium and diammonium salts of EDTA; 1,3-diamino-2-hydroxypropane-N,N,N′,N′-tetraacetic acid; 1,3-diaminopropane-N,N,N′,N′-tetraacetic acid; O,O′-bis(2-aminoethyl)ethyleneglycol-N,N,N′,N′-tetraacetic acid; and 7,19,30-trioxa-1,4,10,13,16,22,27,33-octaazabicyclo[11,11,11]pentatriacontane hexahydrobromide.

Most preferably, the chelators for use in the present invention may include ethylenediamine-N,N,N′,N′-tetraacetic acid (EDTA); the disodium salt of EDTA; 1,3-diaminopropane-N,N,N′,N′-tetraacetic acid; and O,O′-bis(2-aminoethyl)ethyleneglycol-N,N,N′,N′-tetraacetic acid.

In a preferred embodiment this invention provides a preparation (or POEBACA), wherein said chelator in said POEBACA may be selected from the group of chelators consisting of EDTA free acid, EDTA 2Na, EDTA 3Na, EDTA 4Na, EDTA 2K, EDTA 2Li, EDTA 2NH.sub.4, EDTA 3K, Ba(II)-EDTA, Ca(II)-EDTA, Co(II)-EDTA, Cu(II)-EDTA, Dy(III)-EDTA, Eu(III)-EDTA, Fe(III)-EDTA, In(III)-EDTA, La(III)-EDTA, Mg(II)-EDTA, Mn(II)-EDTA, Ni(II)-EDTA, Sm(III)-EDTA, Sr(II)-EDTA, Zn(II)-EDTA, CyDTA, DHEG, DTPA-OH, DTPA, EDDA, EDDP, EDDPO, EDTA-OH, EDTPO, EGTA, HBED, HDTA, HIDA, IDA, Methyl-EDTA, NTA, NTP, NTPO, O-Bistren, and TTHA.

Preferred chelating agents may also be selected from ethylenebis (oxyethylene nitrilio)tetraacetic acid (EGTA) and ethylene diamine tetracetic acid (EDTA), sodium citrate, or oxalate salts such as sodium, potassium, ammonnium or lithium oxalte.

Preferred chelating groups include those derived from polyamino-polycarboxylic groups, e.g. those derived from EDTA, DTPA, DOTA, TETA, TETRA, TITRA or 3,3,9,9-tetramethyl-4,8-diazaundecane-2,10-dione dioxime (HMPAO) or ftom such groups substituted, e.g. by a p-isothiocyanato-phenylC.sub. 1-3 alkyl, preferably p-isothiocyanatobenzyl. Chelating groups derived from DTPA are also preferred.

In a preferred embodiment this invention provides a preparation (or POEBACA), wherein the chelating group is derived from ethylene diaminetetraacetic acid (EDTA), diethylene triamine pentaacetic acid (DTPA), ethylene glycol-O,O′-bis(2-aminoethyl)-N,N,N′,N′-tetraacetic acid (EGTA), N,N′-bis(hydroxybenzyl)ethylenediamine-N,N′-diacetic acid (HBED), triethylenetetramine hexaacetic acid (TTHA), substituted EDTA or -DTPA 1,4,7,10-tetra-azacyclododecane-N,N′,N″,N′″-tetraacetic acid (DOTA) and 1,4,8,11-tetraazacyclotetradecane-N,N′,N″,N′″-tetraacetic acid (TETA), in free form or in pharmaceutically accepted salt form.

In a preferred embodiment this invention provides a preparation (or POEBACA), wherein the chelating group is derived from 1,4,7,10-tetraazacyclotridecane-1,4,7,10-tetraacetic acid (TITRA), 1,4,8,11-tetraazacyclotetradecane (TETRA); EDTA, DTPA, DOTA, TETA, TITRA, TETRA or 3,3,9,9-tetramethyl-4,8-diazaundecane-2,10-dione dioxime (HMPAO) substituted by p-isothiocyanato-phenyl-C.sub. 1-3 alkyl, in free form or in pharmaceutically accepted salt form.

In a preferred embodiment this invention provides a preparation (or POEBACA), comprising R/S-.gamma.-lipoic acid (6,8-dimercaptooctanoic acid) or R/S-.alpha.-lipoic acid (D,L-thioctic acid).

According to separate but non-limiting embodiments of this invention, “substantially enantiomerically pure” 1,2-dithiolane-3-pentanoic acid (thioctic acid, .alpha.-lipoic acid) is within the range from at least about 80% pure to at least about 99% pure inclusive as well as every 1% increment within this range (i.e. at least about 80% pure, at least about 81% pure, at least about 82% pure, etc.).

According to another embodiment of this invention, D,L-thioctic acid can used in the form of the racemic mixture. According to this invention, a racemic mixture can be comprised of two isomers that are found at a ratio within the range from about 20%:80% to about 80%:20% inclusive as well as every 1% increment within this range (i.e. about 20%:80%, about 21%:79%, about 22%:78%, etc.).

According to another embodiment of this invention, optically active R-(+)-.alpha.-lipoic acid is used. R-(+)-.alpha.-lipoic acid is a natural substance that is found in animals and humans, and it acts as coenzyme in the oxidative decarboxylation of .alpha-keto acids.

Microspheres

Specific, but non-limiting, examples of microspheres according to this invention are provided herein. Specific, but non-limiting, examples of ways of making, administering, and using microspheres according to this invention are provided herein. In separate non-limiting embodiments, this invention provides that the micropsheres can be made using lecithin (and/or alternative ingredients as per Table 1 and 2) in amounts in the range from about 0.1 gram to about 40 grams inclusive, including specifically each increment of about 0.1 gram within this range, in a total of 2 ounces of final POEBACA product.

In one embodiment, this invention provides POEBACA comprising gas-filled microspheres. The invention further relates to methods for employing such microspheres as delivery systems to deliver the POEBACAI.

In one embodiment, this invention provides POEBACA comprising at least one member selected from the group consisting of animal and vegetable oils, hydrocarbon oils, ester oils, silicone oils, higher fatty acids, higher alcohols, sunscreening agents, vitamins, alpha lipoic acid, ferulic acid, and flavors and said solid or semi-solid oil component is at least one member selected from the group consisting of animal and vegetable oils, hydrocarbon oils, ester oils, higher fatty acids, higher alcohols, waxes, sunscreening agents and flavors

### EXAMPLE 2

| INGREDIENTS: | per 2 fl oz | | % |
|---|---|---|---|
| Lecithin | 30.0 | gm | 50 |
| EDTA (e.g. Disodium EDTA) | 1.0 | gm | 1.7 |
| Magnesium Chloride | 150.0 | mg | 0.26 |
| Alpha Lipoic Acid | 100.0 | mg | 0.17 |
| Purified Water | | | 37.3 |
| Ethyl Alcohol | | | 10 |
| Gum Arabic | | | 0.5 |

1) Dissolve alpha lipoic acid and EDTA in half the amount of alcohol.

2) Disperse lecithin in half the amount of alcohol and equal amount of water Heat to 50 C, mix with high shear mixing or sonication (sufficient to form micropsheres or liposomes) for 20 minutes, cool to 40 C.

3) Add magnesium chloride and gum arabic to the remaining amount of water, Stir for 30 minutes at room temperature

4) Add step number 3 to step number 2. Mix for 20 minutes

5) Add step 4 to step 1, stir gently for 20 minutes.

6) Take a random samples and test for the presence of liposomes.

### EXAMPLE 3

| INGREDIENTS: | per 2 fl oz | | % |
|---|---|---|---|
| Lecithin | 30.0 | gm | 50 |
| EDTA (e.g. Disodium EDTA) | 1.0 | gm | 1.7 |
| Magnesium Chloride | 150.0 | mg | 0.26 |
| Alpha Lipoic Acid | 100.0 | mg | 0.17 |
| Purified Water | | | 37.3 |
| Ethyl Alcohol | | | 10 |
| Gum Arabic | | | 0.5 |

1) Dissolve alpha lipoic acid in half the amount of alcohol.

2) Disperse lecithin in half the amount of alcohol and equal amount of water Heat to 50° C., mix with high shear mixing or sonication (sufficient to form micropsheres or liposomes) for 20 minutes, cool to 40 C.

3) Add EDTA, magnesium chloride and gum arabic to the remaining amount of water, Stir for 30 minutes at room temperature

4) Add step number 3 to step number 2. Mix for 20 minutes

5) Add step 4 to step 1, stir gently for 20 minutes.

6) Take a random samples and test for the presence of liposomes.

Various modifications of the invention in addition to those shown and described herein will be apparent to those skilled in the art from the foregoing description. Such modifications are also intended to fall within the scope of the appended claims.

In a preferred embodiment this invention provides a preparation (or POEBACA), comprising ocular drug delivery vehicle of an oil-in-water submicron emulsion consisting essentially of about 0.5 to 50% of a first component of an oil, about 0.1 to 10% of a second component of an emulsifier, comprising a phospholipid, about 0.05 to 5% of a non-ionic surfactant and an aqueous component, said submicron emulsion having a mean droplet size in the range of 0.05 to 0.5. mu.m, and a weight ratio of surfactant to oil of about 1:1 or less.

In a preferred embodiment this invention provides a method for transferring ingredients making up a preparation of encapsulated bioavailable chelating agents (i.e. POEBACAI) across a cellular membrane by encapsulating said ingredients within liposomes and carrying said POEBACAI to the cellular membrane where the liposomes will be taken up by the cells, thereby transferring the POEBACAI across the cellular membrane. POEBACAI can be introduced into the interior of a cell of a living organism wherein the liposomes will be decomposed, releasing the POEBACAI to the interior of the cell. The released POEBACAI will complex intracellularly deposited toxic heavy metals, permitting the more soluble metal complex to transfer across the cellular membrane from the cell and subsequently be removed from the living organism.

In a preferred embodiment this invention provides a method of transferring POEBACAI across a cellular membrane comprising: encapsulating said POEBACAI within liposomes; and carrying said liposome encapsulated POEBACAI to said cellular membrane, whereby said liposome encapsulated POEBACAI will transfer across said cellular membrane.

In a preferred embodiment this invention provides a method of introducing a POEBACAI into the interior of a cell in accordance with the method of claim 1 wherein said cellular membrane is the membrane wall of said cell and said encapsulated POEBACAI passes through the membrane wall of said cell into the interior of said cell, wherein said liposomes will be decomposed, thereby releasing said POEBACAI to the interior of said cell.

In a preferred aspect this invention provides a method wherein said cell is a cell of a living organism and said POEBACAI is carried to said cell by injecting a saline suspension of said liposome POEBACAI into the blood stream of said living organism whereby said POEBACAI is carried to the cell within the blood

In a preferred embodiment this invention provides a method for the removal of intracellularly deposited toxic heavy metals comprising:

encapsulating a POEBACAI agent within liposomes;

introducing said liposomal POEBACAI into the blood system by one or more of the following routes: oral administration, intravenous injection, transdermal patch; whereby

said liposome POEBACAI is carried to said body cells within said blood system;

said liposome POEBACAI is passed through the cell wall into the interior of said body cell;

said POEBACAI is released to the interior of said cell by the biological degradation of said liposome by lysosomal enzymes, said released POEBACAI complexing said intracellularly deposited toxic metal;

said complexed toxic metal is passed through the cell wall into said blood stream; and

said complexed toxic metal is removed from said blood stream and the body by normal body processes.

In a preferred embodiment this invention provides a preparation or POEBACA wherein said liposomes are prepared from a mixture of lecithin and cholesterol.

In a preferred embodiment this invention provides a POEBACAI comprised of a member chosen from the group consisting of EDTA, EGTA, and DTPA.

In a preferred embodiment this invention provides a detoxification method wherein said toxic heavy metals are selected from the group consisting of plutonium, gold, mercury, and lead, beryllium, and cadmium.

Any gel can be used in the practice of the present invention. The materials which can be used to form such gels include but are not limited to: carbohydrates such as cellulosics, methylcellulose, starch and modified starch, agarose, gum arabic, ghatti, karay, tragacanth, guar, locust bean gum, tamarind, carageenan, alginate, xanthan, chickle, collagen, polyacrylamide, polysiloxanes (polyanhydrides, e.g., malic anhydride copolymers, polyacrylates, e.g., hydroxyethylpolymethycrylate polymethylmethacrylate, polyethylethacrylate polymethacrylate, ethylenevinylacetate copolymers, ethylenevinylalcohol copolymers, polyorthoesters, .epsilon.-caprolactones, amino acid polymers such as gelled albumin, amino acid polymers and copolymers and gelatins, and other organic or inorganic polymers which can be mixed with liposomes in vitro.

After the mixture forms a gel the resulting liposome-gel matrix can be implanted in tissues. In a particularly useful embodiment of the present invention soft gel matrices such as agarose, collagen and the like containing sequestered liposomes may be injected in vivo. Alternatively, gels such as methylcellulose can be formed in the tissues after inoculation of liposomes in a suspension containing the gel material. After inoculation the suspension forms a gel and the liposomes remain sequestered in the gel matrix rather than dispersed and cleared. Regardless of the method used for preparing and implanting the gel matrix, the release of a liposome entrapped bioactive chelating agent or other POEBACAI is prolonged and the relative concentration of the agent at the site of inoculation is increased.

Virtually any POEBACAI (including chelating agents) as well as virtually any other bioactive agent can be entrapped within the liposomes for use according to the present invention. Such agents include but are not limited to antibacterial compounds, antiviral compounds, antifungal compounds, anti-parasitic compounds, tumoricidal compounds, proteins, toxins, vitamins, trace minerals, heavy metals, enzymes, hormones, neurotransmitters, lipoproteins, glycoproteins, immunoglobulins, immunomodulators, dyes, radiolabels, radio-opaque compounds, fluorescent compounds, polysaccharides, cell receptor binding molecules, anti-inflammatories, antiglaucomic agents, mydriatic compounds, anesthetics, nucleic acids, polynucleotides, etc.

In fact, if concurrent therapy is desired, two or more POEBACAI (including chelating agents) or other bioactive agents may be entrapped in one liposome population which is sequestered in the gel matrix. Alternatively, two or more liposome populations (of the same or different types of liposomes, e.g. mixtures of SPLVs, MPVs, SUVs, LUVs, REVs, etc.) which each entrap the same or different POEBACAI (including chelating agents) or other bioactive chelating agents may be sequestered in the gel matrix.

In yet another embodiment of the present invention the gel can be used as a vehicle for the same or different bioactive chelating agents and other POEBACAI than those entrapped by liposomes.

In certain therapeutic applications it may be desired to deliver a relatively high dose of a drug compound (i.e., compound A) followed by a sustained dose of the same or another compound (i.e., compound B). According to the present invention, this is readily accomplished by entrapping compound B in liposomes, sequestering the liposomes in a gel matrix containing compound A, and administering the same in vivo in a single inoculation. Thus, rapid delivery of compound A by diffusion from the gel, and slow sustained delivery of compound B by release from the liposomes is effected

The release of the bioactive chelating agents may be controlled by the type of liposomes used and the membrane composition of the liposome bilayers as well as by the type and porosity of the gels used. The rate of release is also dependent upon the size and composition of the bioactive chelating agent itself. The liposome itself is the first rate limiting factor in the release of entrapped bioactive chelating agents. The rate of release may depend upon the number of bilayers, the size of the liposomes and most importantly the bilayer composition. A number of researchers add “stabilizers” such as sterols, cholesterols and the like to the phospholipid bilayers in order to alter the permeability of the liposome (Papahadjopoulos, D., Kimilberg, H. K., 1974, in Progress in Surface Science, ed. S. G. Davison, pp. 141-232, Oxford: Pergamon; Demel, R. A., Bruckdorf, K. R., Van Deenan, L. L., 1972, Biochem. Biophys. Acta, 255:331-347). For the present invention it is important that the stable liposomes will release their contents upon contact with body fluids or culture media The rate of release may be controlled by modifying liposome membranes accordingly using known methods.

Use of the Liposome-Gel Preparation in Living Systems.

The liposome-gel compositions of the present invention may be used for sustained delivery of a bioactive chelating agent to cells and/or fluids in vivo and in vitro.

When used in vivo, the liposome-gel compositions of the present invention may be administered before or after gel formation. Routes of administration include but are not limited to: inoculation or injection, (e.g., intraperitoneal, intramuscular, subcutaneous, intra-aural, intra-articular, intra-mammary, etc.), topical application (e.g., on areas, such as eyes, ears, skin or on afflictions such as wounds, burns, etc.), and by absorption through epithelial or mucocutaneous linings (e.g. vaginal and other epithelial linings, gastrointestinal mucosa, etc.).

For example the liposome-gel preparations of the present invention may be inoculated in vivo to provide for the sustained systemic release of the bioactive chelating agent. Such applications may be particularly useful for the systemic release of drugs such as hormones (e.g., to control growth, fertility, sugar metabolism, etc.) or antimicrobials to control and treat infections, etc.

In an alternative example, the liposome-gel preparation may be applied topically. Topical application may be particularly useful for the treatment of wounds (either surgical or non-surgical wounds) where the sustained release of POEBACAI (including chelating agents), antimicrobials and/or blood clotting factors may be helpful in the healing process. Similarly, the liposome-gel preparation may be topically applied to burns for the sustained release of POEBACAI (including chelating agents), antimicrobials and/or cell growth factors. The liposome-gel preparation may also be applied in the ear to treat infections by providing sustained release of POEBACAI (including chelating agents), antimicrobials; this would reduce the necessity of repeated applications of the bioactive chelating agent in the form of ear drops.

In another alternative embodiment, a liposome-gel preparation may be administered orally for sustained release. Such application may be useful for sustained release to oral epithelium and other oral tissues and for sustained release to epithelia of the alimentary tract.

The liposome-gel preparations of the present invention may also be used in vitro to provide for sustained release of a POEBACAI (including chelating agents) into the cell or tissue culture medium. Such POEBACAI (including chelating agents) may also include but are not limited to nutrients, drugs, hormones, growth factors, etc. The liposome-gel preparation may be used as a support for cell adhesion and growth; for instance, a liposome-collagen gel may be especially useful for culturing muscle cells, nerve cell, or liver cells. When the liposome-gel preparation is applied as an overlay, a liposome-agarose gel may be particularly useful.

### References

Many methods for the preparation of micropsheres or liposomes are many in the art. For particularly useful references regarding liposome preparation, see U.S. patents listed below.

The following US patents are incorporated herein in their entirety: U.S. Pat. Nos. 5,000,887; 4,994,213; 4,981,692; 4,975,282; 4,963,297; 4,952,405; 4,944,948; 4,927,637; 4,927,571; 4,923,854; 4,906,476; 4,897,384; 4,895,719; 4,891,208; 4,885,172; 4,880,635; 4,873,088; 4,861,580; 4,839,175; 4,837,028; 4,828,837; 4,822,777; 4,818,537; 4,804,539; 4,781,871; 4,766,046; 4,762,915; 4,752,425; 4,737,323; 4,721,612; 4,714,571; 4,708,861; 4,698,299; 4,668,638; 4,666,831; 4,610,868; 4,588,578; 4,564,599; 4,522,803; 4,483,929 which are all incorporated by reference in their entirety.

Additional references that are also incorporated herein in their entirety include U.S. Pat. Nos.: 5,990,153; 3,932,657;

1. A preparation of encapsulated bioavailable chelating agents comprised of the following ingredients:
a) one or more members selected from a first group consisting of: R-(+)-.alpha.-lipoic acid, S-(−)-.alpha-lipoic acid, R/S-.alpha.-lipoic acid, R/S-.gamma.-lipoic acid, other isomers of alpha lipoic acid, dihydrolipoic acid or DHLA, animal and vegetable oils, hydrocarbon oils, ester oils, silicone oils, higher fatty acids, higher alcohols, sunscreening agents, vitamins, and ferulic acid; wherein at least 10% of said one or more members from said first group is in microspheres or liposomes; and
b) one or more members selected from a second group consisting of: EDTA, EGTA, DPTA, TTHA, HEDHA, NOTA, DOTA, HEDTA, other polyaminopolycarboxylic acids, iminodiacetic acid (IDA), cyclam, penicillamine, dimercaptosuccinic acid, tartrate, thiomalic acid, crown ethers, nitrilotriacetatic acid (NTA), 3,6-dioxaoctanedithioamide, 3,6-dioxaoctanediamide, salicyladoximine, dithio-oxamide, 8-hydroxyquinoline, cupferron, 2,2′-thiobis(ethyl acetoacetate), 2,2′-dipyridyl, and derivatives thereof; wherein at least 1% of said one or more members from said second group is in microspheres or liposomes; wherein at least 10% of said one or more members from said second group is in microspheres or liposomes; and
c) one or more members selected from a third group consisting of: lecithin, phophatidylcholine, phosphatidylserine, phosphatidylethanolamine, dilinoleylphosphatidylcholine, lysolipids, dipalmitoylphosphatidylcholine, distearoylphosphatidylcholine, phosphatidylcholine, phosphatidic acid, sphingomyelin, cholesterol, cholesterol sulfate, cholesterol hemisuccinate, tocopherol hemisuccinate, phosphatidylethanolamine, phosphatidylinositol, fatty acids, palmitic acid, stearic acid, oleic acid, linolenic acid, limoleic acid, glycosphingolipids, glucolipids, glycolipids, sulphatides, lipids bearing sulfonated mono-, di-, oligo- or polysaccharides, lipids with ether and ester-linked fatty acids, triglycerides, lipoproteins, cholesterol, and other lipids or polymerized lipids; wherein at least 10% of said one or more members from said third group is in microspheres or liposomes; and
wherein said or more members selected from said first group, and said or more members selected from said second group members, and said or more members selected from said third group have been admixed and subjected to a procedure to create micropsheres or liposomes.

2. A preparation of encapsulated bioavailable chelating agents comprised of the following ingredients:
a) one or more members selected from a first group consisting of: R-(+)-.alpha.-lipoic acid (substantially enantiomerically pure), S-(−)-.alpha.-lipoic acid (substantially enantiomerically pure), R/S-.alpha.-lipoic acid (racemic mixture), R/S-.gamma.-lipoic acid (racemic mixture), other isomers of alpha lipoic acid, derivatives of alpha lipoic acid (such dihydrolipoic acid or DHLA); wherein at least 25% of said one or more members from said first group is in microspheres or liposomes; and
b) one or more members selected from a second group consisting of: EDTA, EGTA, DPTA, TTHA, HEDHA, NOTA, DOTA, HEDTA, other polyaminopolycarboxylic acids, iminodiacetic acid (IDA), cyclam, penicillamine, dimercaptosuccinic acid, tartrate, thiomalic acid, crown ethers, nitrilotriacetatic acid (NTA), 3,6-dioxaoctanedithioamide, 3,6-dioxaoctanediamide, salicyladoximine, dithio-oxamide, 8-hydroxyquinoline, cupferron, 2,2′-thiobis(ethyl acetoacetate), 2,2′-dipyridyl; wherein at least 25% of said one or more members from said second group is in microspheres or liposomes; and
c) one or more members selected from a third group consisting of: lecithin, phophatidylcholine, phosphatidylserine, phosphatidylethanolamine, dilinoleylphosphatidylcholine, lysolipids, dipalmitoylphosphatidylcholine, distearoylphosphatidylcholine, phosphatidylcholine, phosphatidic acid, sphingomyelin, cholesterol, cholesterol sulfate, cholesterol hemisuccinate, tocopherol hemisuccinate, phosphatidylethanolamine, phosphatidylinositol, palmitic acid, stearic acid, oleic acid, linolenic acid, linoleic acid; wherein at least 25% of said one or more members from said third group is in microspheres or liposomes.
